(19) United States
(12) Patent Application Publication
(10) Pub. No.: US 2021/0395840 A1
(43) Pub. Date: Dec. 23, 2021

(54) METHOD AND A KIT FOR DETECTING PATHOGENS

(71) Applicants: SHIMADZU CORPORATION, Kyoto-shi (JP); National University Corporation Tokyo Medical and Dental University, Tokyo (JP); Nihon Techno Service Co., Ltd., Ushiku (JP); National University Corporation Oita University, Oita City (JP); RIKEN, Wako-shi (JP); Toho University, Tokyo (JP)

(72) Inventors: Masamitsu SHIKATA, Kyoto-shi (JP); Kenji NINOMIYA, Kyoto-shi (JP); Norio SHIMIZU, Tokyo (JP); Hiroshi TAKASE, Tokyo (JP); Manabu MOCHIZUKI, Tokyo (JP); Yasuhiro TOMARU, Ushiku (JP); Satoko NAKANO, Yufu City (JP); Sunao SUGITA, Wako-shi (JP); Takashi SUZUKI, Tokyo (JP)

(73) Assignees: SHIMADZU CORPORATION, Kyoto-shi (JP); National University Corporation Tokyo Medical and Dental University, Tokyo (JP); Nihon Techno Service Co., Ltd., Ushiku (JP); National University Corporation Oita University, Oita City (JP); RIKEN, Wako-shi (JP); Toho University, Tokyo (JP)

(21) Appl. No.: 17/159,783

(22) Filed: Jan. 27, 2021

(30) Foreign Application Priority Data
Jan. 31, 2020 (JP) 2020-015569

(51) Int. Cl. C12Q 1/70; C12Q 1/689; C12Q 1/6893

(52) U.S. Cl. CPC C12Q 1/705; C12Q 1/6893; C12Q 1/689; C12Q 1/70

(57) ABSTRACT

This invention provides a simple analysis method by a PCR method that simultaneously performs, in a single container, separating nucleic acid of pathogens contained in a tissue fragment and preparing a PCR buffer solution, and a kit used for the analysis method. A method for detecting a pathogen includes: (1) obtaining a liquid specimen mixture by adding a tissue fragment containing a pathogen to a PCR buffer solution containing a proteolytic enzyme; (2) heating the liquid specimen mixture at a first temperature; (3) further heating at a second temperature; (4) performing PCR by adding a portion of the liquid mixture obtained in (3) above to a solid composition for PCR reaction containing DNA polymerase and one or more kinds of PCR primer pair; and (5) detecting a PCR product generated in (4) above.

### BACKGROUND OF THE INVENTION

### 1. Field of the Invention

The present invention relates to a method for detecting pathogens in tissue fragments. The present invention also relates to a kit used for detecting pathogens.

### 2. Description of the Related Art

Various methods are used for techniques related to amplification and detection of pathogens. Examples of such a method includes, in addition to PCR methods using polymerase chain reaction (hereinafter, may be referred to as PCR), transcription-reverse transcription concerted reaction (hereinafter, may be referred to as TRC) methods, transcription mediated amplification (hereinafter, may be referred to as TMA) methods, nucleic acid sequence-based amplification (hereinafter, may be referred to as NASBA) methods, loop-mediated isothermal amplification (hereinafter, may be referred to as LAMP) methods, SMart amplification process (hereinafter, may be referred to as SMAP) methods, isothermal and chimeric primer-initiated amplification of nucleic acids (hereinafter, may be referred to as ICAN) methods, and the like.

Among all, the PCR methods are widely applied to research and clinical use due to the advantages of allowing selective amplification of only specific DNA fragments, allowing even an extremely small amount of specimen solution to be processed, relatively short time taken for amplification, simple process of, for example, allowing amplification by a fully automatic desktop device, and the like.

Such a PCR method is used in, for example, diagnosis of uveitis. Uveitis is a generic term for diseases causing inflammation in the eye, and in severe cases, visual impairment, such as loss of sight, occurs at high rates. it is, however, sometimes difficult to differentiate between infectious uveitis and noninfectious uveitis only by clinical findings and there are cases in which the severity of the symptoms increases by a delay in diagnosis or inappropriate treatment.

Examples of the pathogens causing infectious uveitis include viruses, bacteria, fungi, protozoans, and the like, and infectious uveitis caused by virus most frequently occurs. In order to separately detect these pathogens, the PCR method is used which allow rapid detection using a small amount of specimen.

There are kits, each having a single container containing a primer, DNA polymerase, and a probe, used for detecting the presence and the genotype of pathogens in a biological sample collected from a subject by a PCR method (e.g., Japanese Patent No. 6082141). Use of such a kit to dispense nucleic acid purified from the biological sample into the container enables a simple and rapid PCR method.

Such a biological sample collected from a subject, however, contains a large amount of enzyme reaction inhibitors. Since the enzyme reaction inhibitors inhibit PCR, the enzyme reaction inhibitors have to be removed in advance. Pathogens used to be detected by the PCR method after pretreatment, such as removal of the enzyme reaction inhibitors in the biological sample and purification of DNA in the biological sample.

There are also Ampdirect® techniques (e.g., Ann Clin Biochem 37, 674-680 (2000)), which reduce the complexity of the pretreatment, do not inhibit PCR without the pretreatment of the biological sample, and allow detection of pathogens by the PCR method.

### SUMMARY

The above method is generally applied to the case of pathogens in a liquid, such as blood and body fluids. Meanwhile, in the case of pathogens in a tissue fragment, such as cornea, like keratitis, the specimen sample is collected together with the tissue fragment. Accordingly, to analyze the pathogens in the tissue fragment by the PCR method, the nucleic acid of each pathogen first has to be separated from the tissue fragment.

As a method for separating the nucleic acid of a pathogen from a tissue fragment, there is a method in which, since the main component of the tissue fragment is protein, a tissue fragment is added to a buffer solution containing a proteolytic enzyme degrading protein, for example, proteinase K to digest protein in the tissue fragment. However, the proteolytic enzyme degrades protein as well as DNA polymerase, and thus the proteolytic enzyme cannot coexist with DNA polymerase. Although the proteolytic enzyme has to be deactivated before performing PCR, the deactivation may cause deactivation of DNA polymerase when DNA polymerase coexists.

The proteolytic enzyme and DNA polymerase accordingly have to be in separate reactors, and it is not possible to analyze nucleic acid of pathogens by the PCR method by directly adding a tissue fragment, which is a specimen sample, with a PCR buffer solution containing DNA polymerase as the method mentioned above.

A tissue fragment is degraded by the proteolytic enzyme to separate the nucleic acid of pathogens, followed by deactivation of the proteolytic enzyme to deactivate a buffer solution to be added to a PCR buffer solution containing DNA polymerase, and the PCR buffer solution containing DNA polymerase after addition sometimes has modified composition due to the difference in the composition of the buffer solution containing the proteolytic enzyme and the PCR buffer solution containing DNA polymerase and causes the following PCR not to proceed well.

The amount to be added to the PCR buffer solution containing DNA polymerase is thus limited, and in order to increase the amount of the nucleic acid of pathogens, the amount of test fragment used as the specimen sample has to be increased.

It is an object of the present invention to provide a method for simply analyzing nucleic acid of a pathogen contained in a tissue fragment by a PCR method by simultaneously performing, in a single container: separating nucleic acid of a pathogen in a tissue fragment by uniformizing, without separating, a buffer solution containing a proteolytic enzyme used to separate the nucleic acid of the pathogen from the tissue fragment and a PCR buffer solution (by putting the proteolytic enzyme in the PCR buffer solution); and preparing the PCR buffer solution.

That is, the present invention related to a method for detecting a pathogen, including:

(1) obtaining a liquid specimen mixture by adding a tissue fragment containing a pathogen to a PCR buffer solution containing a proteolytic enzyme;

(2) heating the liquid specimen mixture at a first temperature;

(3) further heating at a second temperature;

(4) performing PCR by adding a portion of the liquid mixture obtained in (3) above to a solid composition for PCR reaction containing DNA polymerase and one or more kinds of PCR primer pair; and

(5) detecting a PCR product generated in (4) above.

According to the present invention, it is possible to provide a simple analysis method by a PCR method including simultaneously performing, in a single container, separating nucleic acid of a pathogen contained in a tissue fragment and preparing a PCR buffer solution and also to provide a kit used for the analysis method.

### DETAILED DESCRIPTION OF THE PREFERRED EMBODIMENTS

The method for the present invention is applied to tissue fragments containing pathogens. Such a tissue fragment is cornea or eye mucus.

The pathogens are selected from the group consisting of herpes simplex virus type 1 (HSV-1), varicella zoster virus (VZV), adenovirus (ADV), chlamydia, gonococcus, and acanthamoeba.

The tissue fragments containing the pathogens are rubbed with a cotton swab or the like, and the rubbed tissue fragments containing the pathogens are added to a solution containing a PCR buffer solution and a proteolytic enzyme (hereinafter, may be referred to as a pretreatment solution, and does not contain DNA polymerase) (hereinafter, may be referred to as Step (1)). Immersion of the cotton swab or the like with the tissue fragments containing the pathogens attached in the pretreatment solution allows addition of the tissue fragments containing the pathogens to the pretreatment solution.

The composition of the PCR buffer solution is a tris buffer solution containing KCl, MgCl₂, and dNTP mix (mixture containing dATP, dGTP, dCTP, and dTTP).

KCl preferably has a concentration range from 35 to 75 mM and more preferably approximately 50 mM. MgCl₂ preferably has a concentration range from 1 to 4 mM and more preferably approximately 1.5 mM. The respective concentration ranges of dATP, dGTP, dCTP, and dTTP contained in the dNTP mix are preferably from 50 to 500 μM and more preferably approximately 200 μM.

The amount of the tissue fragments containing the pathogens to be added is preferably from 0.5 to 5 mg, more preferably from 0.5 to 3 mg, and even more preferably approximately 1 mg.

The liquid specimen mixture obtained in Step (1) above is heated at a first temperature (hereinafter, may be referred to as Step (2)). Heating at the first temperature causes the proteolytic enzyme to degrade protein. From the perspective of efficient degradation of protein, the first temperature is preferably 37° C. or more and 60° C. or less.

Step (2) may be performed until protein in the tissue fragments is substantially completely degraded and is preferably performed for, for example, from 30 to 60 minutes approximately.

After Step (2), the solution heated at the first temperature is further heated at a second temperature (hereinafter, may be referred to as Step (3)). Heating at the second temperature causes the proteolytic enzyme to be deactivated. From the perspective of efficient deactivation of the proteolytic enzyme, the second temperature is preferably 90° C. or more and 95° C. or less.

Step (3) may be performed until the proteolytic enzyme is substantially completely deactivated and is preferably performed, for example, from five to 10 minutes approximately.

A portion of the solution heated at the second temperature in Step (3) is added to a solid composition for PCR reaction containing separately prepared DNA polymerase and one or more kinds of PCR primer pair to perform PCR reaction (hereinafter, may be referred to as Step (4)).

Step (4) above amplifies DNA of the pathogens, which is a specimen subject. The DNA polymerase is thermostable DNA polymerase derived from a thermophilic bacterium and examples of the DNA polymerase include Taq, Tth, KOD, Pfu, and variants thereof. From the perspective of avoiding nonspecific amplification by the DNA polymerase, hot start DNA polymerase may be used. Examples of the hot start DNA polymerase include DNA polymerase to which an anti-DNA polymerase antibody binds or DNA polymerase in which an enzyme active site is thermosensitively and chemically modified, and DNA polymerase to which an anti-DNA polymerase antibody binds is preferred.

From the perspective of simultaneous detection of DNA of a plurality of pathogens, a preferred method includes: preparing two or more solid compositions for PCR reaction containing one or more kinds of PCR primer pair; and adding a portion of the solution with the deactivated proteolytic enzyme obtained in Step (3) above to the respective solid compositions for PCR reaction. For example, the solution with the deactivated proteolytic enzyme obtained in Step (3) above is added to the plurality of solid compositions for PCR reaction containing the PCR primer pair to perform PCR, thereby allowing simultaneous detection of DNA of a plurality of pathogens.

Examples of the PCR primer pair used in Step (4) include PCR primer pairs to detect herpes simplex virus type 1 (HSV-1), varicella zoster virus (VZV), adenovirus (ADV), chlamydia, gonococcus, and acanthamoeba.

The solid compositions for PCR reaction mentioned above may contain a PCR primer obtained by combining two or more kinds. This allows detection of DNA of two or more kinds of pathogen using one solid composition for PCR reaction. From the perspective of rapid detection, it is preferred to use a solid composition for PCR reaction obtained by combining two or more kinds of PCR primer pair.

As a method for using a smaller amount of the specimen to simultaneously amplify DNA of a plurality of pathogens, multiplex PCR is proposed (Sugita S, et al. Br J Ophthalmol. 2008; 92:928-932. and Sugita S, et al. Ophthalmology. 2013; 120:1761-1768). The multiplex PCR is a method for simultaneously amplifying a plurality of gene regions using a plurality of PCR primer pairs in one PCR reaction system. This method has an advantage of, in addition to use of a smaller amount of the specimen, simultaneous detection of a plurality of pathogens. In this method, however, the nucleic acid has to be extracted from the specimen before performing PCR. In the multiplex PCR, the primers to be used have to be set and the reaction conditions have to be investigated to satisfactorily proceed with amplification of the target gene regions by the respective PCR primer pairs in one PCR reaction system.

The solid composition for PCR reaction used in Step (4) above is generally prepared by freeze drying while the preparation method is not limited to freeze drying as long as the activity of the enzyme and the like contained in the solid composition is maintained. The form of a solid composition allows PCR to be started only by adding the solution with the deactivated proteolytic enzyme obtained in Step (3) above, thereby simplifying the measurement operation. It also simplifies storage before use.

The solid composition for PCR reaction in Step (4) above preferably contain an oligonucleotide probe labeled with one or more kinds of fluorescent dye to fluorescently detect a PCR amplification product from the perspective of detection accuracy. When the solid composition for PCR reaction contains one kind of PCR primer pair, a fluorescent dye may be one kind for real-time measurement as described later. In contrast, when the solid composition for PCR reaction contains two or more kinds of PCR primer pair, two or more kinds of fluorescent dye different from each other has to be used. Examples of the fluorescent dye include 6-carboxyfluorescein (hereinafter, may be referred to as FAM), 6-carboxy-X-rhodamine (hereinafter, may be referred to as ROX), a cyanine-based dye (hereinafter, may be referred to as Cy5), and 4,7,2′,4′,5′,7′-hexachloro-6-carboxyfluorescein (hereinafter, may be referred to as HEX). The base sequence of the oligonucleotide probe may be appropriately designed based on base sequence information in sequence database (GenBank, etc.) of the PCR amplification product.

By adding the solution heated at the second temperature obtained in Step (3) to the solid composition for PCR reaction described above, the solid composition for PCR reaction is dissolved and thermal cycling is performed to proceed with PCR. The PCR conditions (temperature, time, and the number of cycles) are appropriately set according to the kind of DNA of the expected pathogens and the like. When the PCR proceeds and the DNA of the pathogens is contained in the solution heated at the second temperature obtained in Step (3), a positive result is obtained in detecting of a PCR product described later to perform disease diagnosis, assessment of disease incidence risk, and the like.

A PCR product generated as a result of Step (4) above is detected (hereinafter, may be referred to as Step (5)).

Examples of the method for detecting the PCR product include electrophoresis using agarose gel, detection by a thermal melting curve, fluorescence detection, and the like. From the perspective of rapid detection, a detection method called as real-time measurement is preferred.

The real-time measurement of a PCR product is also called as real-time PCR. In real-time PCR, a PCR amplification product is generally detected by fluorescence. Examples of the fluorescence detection method include a method using an intercalator fluorescent dye and a method using a fluorescently labeled probe. An example of the intercalator fluorescent dye includes SYBR® Green I. The intercalator fluorescent dye binds to double-stranded DNA synthesized by the PCR and emits fluorescence by irradiation with excitation light. Measurement of the fluorescence intensity allows measurement of the amount of the generated PCR amplification product.

Examples of the fluorescently labeled probe include a hydrolysis probe, a molecular beacon, a cycling probe, and the like. The hydrolysis probe is oligonucleotide having a 5′ end modified with a fluorescent dye and a 3′ end modified with a quenching material. Although the hydrolysis probe is specifically hybridized with template DNA in an annealing of PCR, the presence of the quencher on the probe inhibits generation of fluorescence even by irradiation with excitation light. In elongation reaction after that, for example when the hydrolysis probe hybridized with the template DNA is degraded by 5′->3′ exonuclease activity of Taq DNA polymerase, the fluorescent dye is removed from the probe and the inhibition of the fluorescence generation by the quencher is released to emit fluorescence. Measurement of the fluorescence intensity allows measurement of the amount of the generated amplification product.

Examples of the fluorescent dye include fluorescent dyes similar to those described above. Examples of the quencher include TAMRA®, Black Hole Quencher (BHQ)® 1, BHQ 2, MGB-Eclipse®, DABCYL, and the like. To distinctively detect two or more kinds of target nucleic acid, two or more kinds of oligonucleotide probe (e.g., hydrolysis probe) labeled with respectively different fluorescent dyes are preferably used for PCR from the perspective of detection accuracy.

For real-time measurement of a PCR product, the amplification curve of the PCR product is monitored using a fluorescent filter corresponding to the fluorescent dye to be used, thereby allowing real-time checking of the progress of PCR. When the fluorescence intensity increases with the number of PCR cycles, the presence of DNA of the pathogens is assessed as positive. In contrast, when the fluorescence intensity does not increase, the presence is assessed as negative.

The PCR method is pointed out to increase the possibility of a human error, such as using a wrong reagent, and cause a false detection result. False positive and the like may occur by, other than such a human error, mixing (contamination) of an amplification product of previous nucleic acid amplification reaction in a container for new nucleic acid amplification reaction.

In amplification and detection of nucleic acid, a result sometimes comes out as negative (false negative) for some reason though it is actually positive. Such a false negative result means nucleic acid that should have been detected is not detected and thus false negative has to be prevented as much as possible.

From the perspective of preventing false positive and false negative described above, the method for detecting pathogens of the present invention preferably further includes: performing PCR by adding a portion of the liquid mixture obtained in Step (3) above to a solid composition for PCR control containing DNA polymerase, positive control nucleic acid, and PCR reaction control nucleic acid (hereinafter, may be referred to as Step (6)); and detecting a PCR product generated in Step (6) above (hereinafter, may be referred to as Step (7)).

The positive control nucleic acid used in Step (6) above is also useful for quantification (absolute quantification or relative quantification) of the pathogen subject. When the positive control nucleic acid is used for absolute quantification, for example, preparation of a calibration curve based on a result of measuring the positive control nucleic acid with a known concentration allows accurate quantification of the pathogen with an unknown concentration. When the positive control nucleic acid is used for relative quantification, for example, the number of cycles for reaching a certain concentration may be compared between positive control nucleic acid and the pathogen subject to calculate a relative concentration difference based on the PCR principle of double amplification in one cycle.

The positive control nucleic acid may be extracted and amplified in advance from the pathogen subject, or may be separately extracted from different species. The positive control nucleic acid may be artificially synthesized nucleic acid.

The positive control nucleic acid is preferably nucleic acid considered to be contained in the specimen pathogen, and from the perspective of the above effects, more preferably a housekeeping gene.

Examples of the housekeeping gene include a TATA-binding protein (hereinafter, may be referred to as TBP) gene, a glyceraldehyde-3-phosphate dehydrogenase (hereinafter, may be referred to as GAPDH) gene, a β-actin gene, a β2-microglobulin gene, hypoxanthine phosphoribosyl transferase 1 (hereinafter, may be referred to as HPRT 1), a 18S rRNA gene, a 5-aminolevulinate synthase (hereinafter, may be referred to as ALAS) gene, a β-globin gene, a glucose-6-phosphate dehydrogenase (hereinafter, may be referred to as G6PD) gene, a β-glucuronidase (hereinafter, may be referred to as GUSB) gene, an importin 8 (hereinafter, may be referred to as IPO8) gene, a porphobilinogen deaminase (hereinafter, may be referred to as PBGD) gene, a phosphoglycerate kinase 1 (hereinafter, may be referred to as PGK1) gene, a peptidylprolyl isomerase A (hereinafter, may be referred to as PPIA) gene, a ribosomal protein L13a (hereinafter, may be referred to as RPL13A) gene, a ribosomal protein large PO (hereinafter, may be referred to as RPLP0) gene, a succinate dehydrogenase subunit A (hereinafter, may be referred to as SDHA) gene, a transferrin receptor (hereinafter, may be referred to as TFRC) gene, a 3-monooxygenase/tryptophan 5-monooxygenase activation protein, zeta (hereinafter, may be referred to as YWHAZ) gene, and the like.

The PCR reaction control nucleic acid used in Step (6) above exhibiting a positive amplification curve is an index representing that the analysis is correctly performed, that is, the pathogen subject is correctly added to the above solid composition for PCR reaction.

The PCR reaction control nucleic acid may be extracted and amplified in advance from the pathogen subject, or may be separately extracted from different species. The PCR reaction control nucleic acid may be artificially synthesized nucleic acid.

The PCR reaction control nucleic acid is preferably nucleic acid contained in the pathogen subject and more preferably a housekeeping gene from the perspective of the above effects.

Examples of the housekeeping gene include nucleic acids same as those for the positive control nucleic acid while nucleic acid different from the positive control nucleic acid is preferably used from the perspective of checking that the pathogen subject is correctly added to the solid composition for PCR reaction.

The solid composition for PCR control used in Step (6) above is generally prepared by freeze drying similar to the PCR solid composition described above while the preparation method is not limited to freeze drying as long as the activity of the enzyme and the like contained in the solid composition is maintained. The form of a solid composition allows PCR to be started only by adding the solution with the deactivated proteolytic enzyme obtained in Step (3) above, thereby simplifying the measurement operation. It also simplifies storage before use.

Examples of the method for detecting the PCR product in Step (7) above, similar to Step (5) above, include electrophoresis using agarose gel, detection by a thermal melting curve, fluorescence detection, and the like. From the perspective of rapid detection, a detection method called as real-time measurement is preferred. The methods for detecting the PCR product in Steps (5) and (7) are preferably the same for the simplification of the operation.

For example, in the case of both Steps (5) and (7) performed by real-time PCR, when the fluorescence intensity of the positive control nucleic acid increases with the number of PCR cycles, the pathogen is determined to be correctly served for Step (4). In addition, when the fluorescence intensity of the PCR reaction control nucleic acid increases with the number of PCR cycles, it is determined that the pathogen is correctly served for Step (4) and also the DNA polymerase and the PCR primer pair are normally functioning. The reliability of the assessment that the presence of the pathogen is negative is thus improved.

From the perspective of detection precision, the combination of the positive control nucleic acid and the PCR reaction control nucleic acid is preferably a combination of GAPDH and TBP. GAPDH is commonly expressed as a housekeeping gene in a certain amount in many tissues and cells and is used as a positive control to check the progress of PCR. TBP is a general transcription factor binding to a DNA sequence called as a TATA box and reflects the number of cells and thus is used as a PCR reaction control to check that the cells are collected and contained in the specimen.

To efficiently perform the detection method, the present invention further provides a kit for testing a pathogen including (1) and (2) below:

(1) a specimen collection container containing a PCR buffer solution and a proteolytic enzyme; and

(2) at least one or more PCR reaction containers, each containing a solid composition for PCR reaction containing DNA polymerase and one or more kinds of PCR primer pair.

The testing kit allows efficient testing when a very small amount of the specimen is collected to test a plurality of target nucleic acids in accordance with the procedure described above.

The testing kit of the present invention includes a specimen collection container containing a PCR buffer solution and a proteolytic enzyme. The contained PCR buffer solution and the contained proteolytic enzyme are as described above. The specimen collection container is not particularly limited in shape, size, and the like and is preferably made of a material convenient in handling and excellent in chemical resistance. In addition, a preferred material is excellent in visibility. From the perspective of ease of handling, a container with a lid is preferred.

A cotton swab or the like with which the tissue fragments containing the pathogens are rubbed is immersed in the specimen collection container, and the tissue fragments containing the pathogens are mixed with the solution in the specimen collection container to obtain a liquid specimen mixture. The specimen collection container containing the liquid specimen mixture thus obtained is directly heated at the first temperature, followed by heating at the second temperature.

The testing kit of the present invention includes at least one or more PCR reaction containers containing a solid composition for PCR reaction containing DNA polymerase and one or more kinds of PCR primer pair. A portion of the solution heated at the second temperature in the specimen collection container is collected and an appropriate amount of the solution is dropped into the PCR reaction container to perform thermal cycling as described above and proceed with PCR reaction in the PCR reaction container, and if there are pathogens in the specimen, the pathogens are amplified.

The DNA polymerase and the one or more kinds of PCR primer pair contained in the solid composition for PCR reaction are as described above. The solid composition for PCR reaction is also as described above.

The PCR reaction container may contain the solid composition for PCR reaction containing the oligonucleotide probe labeled with one or more kinds of fluorescent dye to fluorescently detect the PCR amplification product. From the perspective of reducing the amount of the specimen, the solid composition for PCR reaction is preferably used that contains a plurality of PCR primer pairs and/or two or more kinds of fluorescent dye.

The number of PCR reaction container(s) is appropriately set according to the numbers of the kinds of specimen pathogen and PCR primer pair contained in the solid composition for PCR reaction. From the perspective of the ease of handling, each container preferably has a lid.

From the perspective of preventing false positive and false negative, the testing kit of the present invention may further include a PCR reaction control container that contains a solid composition for PCR control containing DNA polymerase, positive control nucleic acid, and PCR reaction control nucleic acid. A portion of the solution heated at the second temperature in the specimen collection container is collected and an appropriate amount of the solution is dropped into the PCR reaction control container to perform thermal cycling as described above and proceed with PCR reaction in the PCR reaction control container, and the positive control nucleic acid and the PCR reaction control nucleic acid are amplified.

The DNA polymerase, the positive control nucleic acid, and the PCR reaction control nucleic acid contained in the solid composition for PCR reaction control are as described above. The solid composition for PCR reaction control is also as described above.

The number of the PCR reaction control containers may be one, two, or more. From the perspective of the convenience of the operation, the number of the PCR reaction control container is preferably one. In contrast, from the perspective of increasing the accuracy of PCR reaction results, the number of the PCR reaction control containers is preferably two or more. The number of the PCR reaction control containers is appropriately set according to the number of the kinds of target nucleic acid, the amount of the specimen, and the like. The PCR reaction control container may contain the solid composition for PCR control containing the oligonucleotide probe labeled with one or more kinds of fluorescent dye to fluorescently detect the PCR amplification product.

The PCR product obtained by the testing kit is used for an analysis method, such as electrophoresis, detection by a thermal melting curve, and fluorescence detection, and analysis.

The specimen collection container, the PCR reaction container, and the PCR reaction control container may be same or different from each other in material, shape, volume, and the like. A preferred material is easily handled and excellent in chemical resistance. The preferred material is also excellent in visibility. Examples of the material include glass, polypropylene, and the like.

From the perspective of ease of handling, for example, all containers are preferably same in shape, volume, and the like. In addition, from the perspective of suppressing human errors such as omissions of adding the specimen and the respective mixtures, each container is preferably prepared to allow identification with colors, signs, numbers, and the like.

Examples of the respective containers to be used include a tube strip in which a plurality of tubes are coupled, and a tube strip coupled with a well is preferred. The number is generally from two to 12, preferably from two to ten, and more preferably from two to eight.

### EXAMPLES

The present invention is described in detail with reference to Examples while the scope of the present invention is not limited to them.

### Example 1

### Detection of Infectious Keratitis Positive Specimen

Cornea or eye mucus of specimens obtained from patients suspected of infectious uveitis was mixed with 180 μL of a pretreatment solution (PCR buffer solution containing a proteolytic enzyme). The composition of the pretreatment solution after mixing was 200 μg/mL of Proteinase K, 0.05% (w/v) of a nonionic surfactant, 1.5 mM of MgCl₂, 35 mM of KCl, and 200 μM each of dNTP (dATP, dGTP, dCTP, and dTTP). Then, 20 μL of the solution after pretreatment was dispensed into each tube of an eight-tube strip containing a solid composition for PCR reaction. The solid composition for PCR reaction in the strip tube contains DNA polymerase, an oligonucleotide probe labeled with a fluorescent dye to fluorescently detect a PCR amplification product different for each tube, and PCR primer pairs.

As the PCR primer pairs for detecting pathogens, those with the following base sequences.

Primer Pair for Detecting GAPDH Gene
Forward 5′-tgtgctcccactcctgatttc-3′ (Sequence
Number 1)
Reverse 5′-cctagtcccagggctttgatt-3′ (Sequence
Number 2)
Primer Pair for Detecting TBP Gene
Forward 5′-gcaccactccactgtatccc-3′ (Sequence
Number 3)
Reverse 5′-cccagaactctccgaagctg-3′ (Sequence
Number 4)
Primer Pair for Detecting HSV-1
Forward 5′-cgcatcaagaccacctcctc-3′ (Sequence
Number 5)
Reverse 5′-gtcagctcgtgRttctg-3′ (Sequence Number 6)
Target Gene for Amplification: UL27
Primer Pair for Detecting VZV
Forward 5′-tcactaccagtcatttctatccatctg-3′ (Sequence
Number 7)
Reverse 5′-gaaaacccaaaccgttctcgag-3′ (Sequence
Number 8)
Target Gene for Amplification: ORF29
Primer Pair for Detecting Adenovirus
Forward 1 5′-tgggcgtacatgcacatc-3′ (Sequence
Number 9)
Forward 2 5′-gtggtcttacatgcacatc-3′ (Sequence
Number 9)
Forward 3 5′-atggtcttacatgcacatc-3′ (Sequence
Number 9)
Forward 4 5′-tgggcatacatgcacatc-3′ (Sequence
Number 9)
Forward 5 5′-tgggcttacatgcacatc-3′ (Sequence
Number 9)
Reverse 1 5′-cgggcgaactgcacca-3′ (Sequence Number
10)
Reverse 2 5′-cgggcaaactgcacca-3′ (Sequence Number
10)
Reverse 3 5′-cgggcgaattgcacca-3′ (Sequence Number
10)
Reverse 4 5′-cgggcaaattgcacca-3′ (Sequence Number
10)
Reverse 5 5′-cgggcaaactgcacga-3′ (Sequence Number
10)
Target Gene for Amplification:
Primer Pair for Detecting Chlamydia
Forward 5′-gaaaagaacccttgttaagggag-3′ (Sequence
Number 11)
Reverse 5′-cttaactccctggctcatcatg-3′ (Sequence
Number 12)
Target Gene for Amplification:
Primer Pair for Detecting Gonococcus
Forward 5′-ggaaagtaatcagatgaaaccagttc-3′ (Sequence
Number 13)
Reverse 5′-ggatcggtatcactcgctct-3′ (Sequence
Number 14)
Target Gene for Amplification:
Primer Pair for Detecting Acanthamoeba
Forward 1 5′-tcaaagcaggcagatYcaatt-3′ (Sequence
Number 17)
Forward 2 5′-tcaaagcaggcagatttaacca-3′ (Sequence
Number 17)
Reverse 5′-gtcctattccattatcccatgctaa-3′ (Sequence
Number 18)
Target Gene for Amplification:

As the oligonucleotide probe to detect PCR amplified products, those having a 5′ end labeled with fluorescent dye ROX were used. All the oligonucleotide probes used here had a 3′ end modified with a quenching material BHQ. The probes with the following base sequence were used.

Probe for Detecting GAPDH Gene
5′-aaaagagctaggaaggacaggcaacttggc-3′
(Sequence Number 23) (Labeled with ROX)
Probe for Detecting TBP gene
5′-acccccatcactcctgccacgc-3′
(Sequence Number 24) (Labeled with ROX)
Probe for Detecting HSV-1
5′-tggcaacgcggcccaac-3′
(Sequence Number 25) (Labeled with ROX)
Probe for Detecting VZV
5′-tgtctttcacggaggcaaacacgt-3′
(Sequence Number 26) (Labeled with ROX)
Probe for Detecting Adenovirus
5′-caggaYgcYtcggagta-3′
(Sequence Number 27) (Labeled with ROX)
Probe for Detecting Chlamydia
5′-caaaaggcacgccgtcaac-3′
(Sequence Number 28) (Labeled with ROX)
Probe for Detecting Gonococcus
5′-gaaacacgccaatgaggggcatgat-3′
(Sequence Number 29) (Labeled with ROX)
Probe for Detecting Acanthamoeba
5′-ctgccaccgaatac-3′
(Sequence Number 31) (Labeled with ROX)

The PCR reaction in the eight-tube strip containing the solid compositions for PCR reaction dissolved by the PCR buffer solution after processing the specimen was monitored by the hydrolysis probe method using a real-time PCR device. As the PCR conditions, initial denaturation was performed at 95° C./for 10 seconds and then 45 cycles of PCR at 95° C./for 5 seconds-60° C./for 20 seconds. The presence (positive) or the absence (negative) of a pathogenic microorganism as the target was determined based on the Cq value (the number of cycles where the amplification curve intersects with a threshold line). As a comparison, DNA was purified from each specimen, followed by quantification of the number of copies by the real-time PCR (qPCR) method.

The pathogens measured by the method of the present invention were compared with the real-time PCR (qPCR) method. The correlation was examined between the quantitative values by the real-time PCR (qPCR) method and the Cq values measured by the method of the present invention.

From the results of above, the positive specimens quantified by the real-time PCR (qPCR) method were all positive when measured using the method of the present invention as well. In addition, the results indicated that HSV-1, VZV, adenovirus, chlamydia, gonococcus, and acanthamoeba were identified. The results also indicated that the quantitative values were correlated with the Cq values.

### Example 2

### Analysis of Specimens Diagnosed as Noninfectious Uveitis

Specimens obtained from patients diagnosed as noninfectious uveitis were measured by the real-time PCR (qPCR) method and the method of the present invention. All specimens were negative by the real-time PCR (qPCR) method and all were negative by the method of the present invention. That is, the results indicated that the measurement results obtained from both methods coincided with each other.

### Aspects

Those skilled in the art understand that the embodiments described above as exemplifications are specific examples of the following aspects.

[1] A method for detecting a pathogen, including:

(1) obtaining a liquid specimen mixture by adding a tissue fragment containing a pathogen to a PCR buffer solution containing a proteolytic enzyme;

(2) heating the liquid specimen mixture at a first temperature;

(3) further heating at a second temperature;

(4) performing PCR by adding a portion of the liquid mixture obtained in (3) above to a solid composition for PCR reaction containing DNA polymerase and one or more kinds of PCR primer pair; and

(5) detecting a PCR product generated in (4) above.

The invention of [1] above allows providing a simple analysis method by a PCR method that simultaneously performs, in a single container, separating nucleic acid of pathogens contained in a tissue fragment and preparing a PCR buffer solution.

[2] The detection method according to [1] above, wherein the first temperature in (2) above is 37° C. or more and 60° C. or less.

The invention of [2] above allows efficient degradation of protein.

[3] The detection method according to [1] above, wherein the second temperature in (3) above is 90° C. or more and 95° C. or less.

The invention of [3] above allows efficient deactivation of a proteolytic enzyme.

[4] The detection method according to [1] above, wherein the tissue fragment is cornea or eye mucus.
[5] The detection method according to [1] above, wherein the pathogen is selected from the group consisting of herpes simplex virus type 1 (HSV-1), varicella zoster virus (VZV), adenovirus (ADV), chlamydia, gonococcus, and acanthamoeba.

The inventions of [4] and [5] above allow simple detection of various pathogens in cornea or eye mucus.

[6] The detection method according to [1] above, wherein the proteolytic enzyme is proteinase K.

The invention of [6] above allows efficient degradation of protein.

[7] The detection method according to [1] above, wherein the PCR product is measured in (5) above by real-time PCR.

The invention of [7] above allows rapid detection of pathogens.

[8] A kit for detecting a pathogen, including:

(1) a specimen collection container containing a PCR buffer solution containing a proteolytic enzyme; and

(2) at least one or more PCR reaction containers containing a solid composition for PCR reaction containing DNA polymerase and one or more kinds of PCR primer pair.

The invention of [8] above allows providing a kit that is capable of simultaneously performing, in a single container, separating nucleic acid of pathogens contained in a tissue fragment and preparing a PCR buffer solution.

What is claimed is:

1. A method for detecting a pathogen, comprising:
(1) obtaining a liquid specimen mixture by adding a tissue fragment containing a pathogen to a PCR buffer solution containing a proteolytic enzyme;
(2) heating the liquid specimen mixture at a first temperature;
(3) further heating at a second temperature;
(4) performing PCR by adding a portion of the liquid mixture obtained in (3) above to a solid composition for PCR reaction containing DNA polymerase and one or more kinds of PCR primer pair; and
(5) detecting a PCR product generated in (4) above.

2. The detection method according to claim 1, wherein the first temperature in (2) above is 37° C. or more and 60° C. or less.

3. The detection method according to claim 1, wherein the second temperature in (3) above is 90° C. or more and 95° C. or less.

4. The detection method according to claim 1, wherein the tissue fragment is cornea or eye mucus.

5. The detection method according to claim 1, wherein the pathogen is selected from the group consisting of herpes simplex virus type 1 (HSV-1), varicella zoster virus (VZV), adenovirus (ADV), chlamydia, gonococcus, and acanthamoeba.

6. The detection method according to claim 1, wherein the proteolytic enzyme is proteinase K.

7. The detection method according to claim 1, wherein the PCR product is measured in (5) above by real-time PCR.

8. A kit for detecting a pathogen, comprising:
(1) a specimen collection container containing a PCR buffer solution containing a proteolytic enzyme; and
(2) at least one or more PCR reaction containers containing a solid composition for PCR reaction containing DNA polymerase and one or more kinds of PCR primer pair.
